(19) United States
(12) Patent Application Publication
(10) Pub. No.: US 2024/0359145 A1
(43) Pub. Date: Oct. 31, 2024

(54) PURIFICATION METHODS COMPRISING THE USE OF MEMBRANES OBTAINED FROM BIO-BASED SULFONE POLYMERS

(71) Applicant: Solvay SpecialtyPolymers USA, LLC, Alpharetta, GA (US)

(72) Inventors: Emanuele DI NICOLO', Gorla Minore (IT); Pasquale CAMPANELLI, Limbiate (IT); David B. THOMAS, Milton, GA (US)

(21) Appl. No.: 18/764,614

(22) Filed: Jul. 5, 2024

Related U.S. Application Data
(63) Continuation of application No. 16/644,171, filed on Mar. 4, 2020, now abandoned.
(60) Provisional application No. 62/556,636, filed on Sep. 11, 2017.

(30) Foreign Application Priority Data
Oct. 3, 2017 (EP) 17194549.6

(51) Int. Cl. B01D 71/68; A61M 1/16; B01D 61/24; B01D 69/02; B01D 71/52; C08L 81/06

(52) U.S. Cl. CPC B01D 71/68; A61M 1/1621; B01D 61/243; B01D 69/02; B01D 71/522; C08L 81/06; B01D 2325/36; C08L 2201/56

(57) ABSTRACT

A purification method for a biological fluid comprising at least a filtration step through a membrane obtained from a sulfone polymer (PSI) derived from bio-based feed-stocks. In particular the polymer (PSI) comprises more than 50% moles recurring units (R_{PSI}) comprising sugar moieties selected from the group consisting of those of formulae (E′-I) to (E′-III):

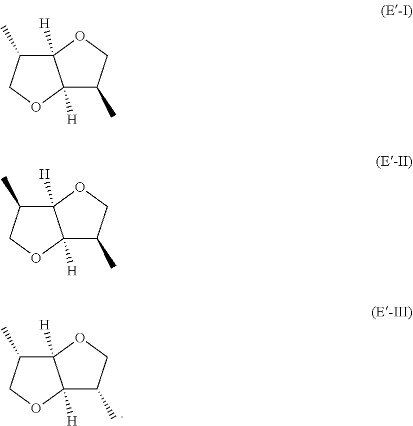

A membrane free from pore-forming agents comprising at least one sulfone polymer (PSI) and a method for preparing such a membrane.

### RELATED APPLICATIONS

This application claims priority to U.S. provisional application No. 62/556,636 filed on Sep. 11, 2017 and to European patent application No. 17194549.6 filed on Oct. 3, 2017, the whole content of each of these applications being incorporated herein by reference for all purposes.

### TECHNICAL FIELD

The present invention relates to purification methods comprising the use of membranes obtained from specific polyarylene ether sulfones derived from bio-based feed-stocks, in particular to methods for purifying biological fluids.

### BACKGROUND ART

Aromatic sulfones polymers are high performance polymers endowed with high mechanical strength and high thermal stability; they are used in a variety of industrial and commercial applications, including the manufacture of microfiltration membranes and ultrafiltration membranes, such as those used in the biomedical field. For example, micro-porous membranes used in the manufacture of haemodialysis devices can be obtained by spinning filaments from a dope solution (otherwise referred to as “spinning solution”) comprising the polymer, a solvent, a pore-forming agent and a surface-modifying macromolecule, as disclosed, for example, in US 2011/009799 A (INTERFACE BIOLOGICS, INC.), published on Jan. 13, 2011.

In particular, aromatic sulfone polymers having para-linked diphenylenesulfone groups as part of their backbone repeat units are a class of thermoplastic polymers characterized by high glass-transition temperatures, good mechanical strength and stiffness, and outstanding thermal and oxidative resistance. Also these polymers are suitable for an increasingly wide and diversified range of commercial applications, including notably the manufacture of coatings and membranes.

Among aromatic sulfones polymers, polyarylene ether sulfones derived from bio-based feed-stocks have been described in the art, as part of efforts oriented towards the reduction of the amount of petroleum consumed in the chemical industry and to open new high-value-added markets to agriculture; 1,4:3,6-dianhydrohexitols are examples of such chemicals used as bio-based feed-stock, which, by virtue of their bicyclic constrained geometry and their oxygenated rings, can provide advantageous features when incorporated into a polyarylene ether sulfone.

Depending on the chirality, three isomers of the 1,4:3,6-dianhydrohexitols sugar diol exist, namely isosorbide (1), isomannide (2) and isoidide (3):

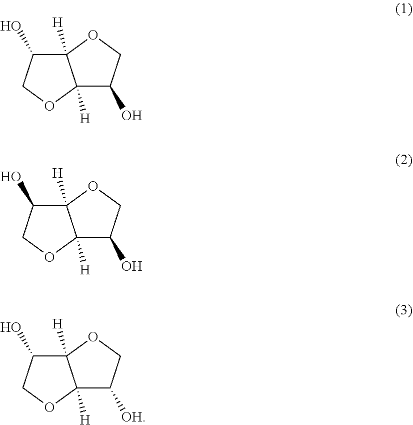 [SMILES: O[C@@H]1CO[C@H]2[C@@H]1OC[C@@H]2O ; O[C@@H]1CO[C@H]2[C@@H]1OC[C@H]2O ; O[C@H]1CO[C@H]2[C@@H]1OC[C@@H]2O]

The 1,4:3,6-dianhydrohexitols are composed of two cis-fused tetrahydrofuran rings, nearly planar and V-shaped with a 120° angle between rings. The hydroxyl groups are situated at carbons 2 and 5 and positioned on either inside or outside the V-shaped molecule. They are designated, respectively, as endo or exo. Isoidide (1) has two exo hydroxyl groups, whereas in isomannide (2) they are both endo, and in isosorbide (3) there is one exo and one endo hydroxyl group. It is generally understood that the presence of the exo substituent increases the stability of the cycle to which it is attached. Also, exo and endo groups exhibit different reactivities since they are more or less accessible depending on the steric requirements of the studied reaction. The reactivity also depends on the existence of intramolecular hydrogen bonds.

Within this frame, Kricheldorf et al. first reported the preparation and characterization of poly(ether sulfone)s containing isosorbide moieties in 1995 (H. Kricheldorf, M. Al Masri, J. Polymer Sci., Pt A: Polymer Chemistry 1995, 33, 2667-2671), although of limited molecular weight and through complex synthetic routes. More recent developments have made available poly ether sulfones comprising isosorbide groups through simpler and more effective synthetic methods, so delivering materials of higher molecular weight through an approach which can be scaled up to industrial level. Hence, WO 2014/072473 (SOLVAY SPECIALTY POLYMERS USA, LLC) 15/05/2014 provides an improved method of making poly(arylether sulfone) polymers from 1,4:3,6-dianhydrohexitol and certain dihaloaryl compounds which enables obtaining polymers having increased molecular weight. Polysulfone isosorbide materials described therein are taught as notably useful for the manufacture of membranes, although no specific example of the actual manufacture of membranes, and more specifically of hollow fiber membranes, is provided.

Manufacturing techniques for the industrial production of membranes generally include the preparation of solutions of polyaryl ether sulfone polymers in suitable solvents, possibly in combination with specific pore forming agents. According to these techniques, a clear polymer solution, often referred to as “dope” or “dope solution”, is precipitated into two phases: a solid, polymer-rich phase that forms the matrix of the membrane, and a liquid, polymer-poor phase that forms the membrane pores. Polymer precipitation from a solution is generally induced by contacting the dope solution with a non-solvent, causing hence coagulation of the polymer. As pore forming agents, polyvinylpyrrolidone (PVP), and polyethyleneglycol (PEG) are typically used. When PVP is used, it is preferred to use high molecular weight PVP, such as K30, K85 and K90, such as those available from Basf. Although membranes are usually subjected to a final washing step, a certain amount of pore-forming agent remains in the membrane. However, for membranes used in the filtration of blood though extracorporeal circuits, namely through haemodialyzers, it would be desirable to reduce as much as possible the amount of pore-forming agents, in particular that of PVP, as it may cause allergic reactions in patients and may also undergo degradation during sterilization of the membranes.

A further crucial requirement is that materials used for the manufacture of blood filtration membranes must not induce blood coagulation. Indeed, in patients undergoing chronic haemodialysis, i.e. more haemodialysis sessions for prolonged hours, heparin is administered in order to avoid blood coagulation and clogging of the membrane. However, heparin may cause allergic reactions and may also interfere with other medical treatments that a patient might be taking. Prolonged use of heparin may also cause bleeding and hypertriglyceridemia.

### SUMMARY OF INVENTION

The invention thus pertains to purification methods [method (MPUR)] for biological fluids comprising at least one filtration step through a membrane [membrane (ME)] obtained from at least one sulfone polymer [polymer (PSI)], said polymer (PSI) having recurring units, wherein more than 50% moles, with respect to all the recurring units of polymer (PSI), are recurring units (R_{PSI}) selected from the group consisting of those of formulae (R_{PSI}-1) and (R_{PSI}-2) herein below:

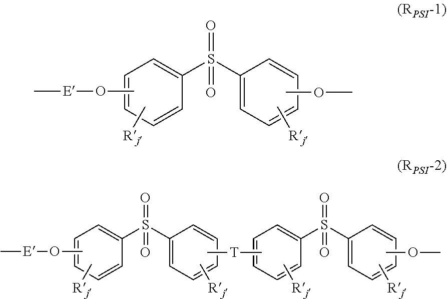

wherein
each of E′, equal to or different from each other and at each occurrence, is selected from the group consisting of those of formulae (E′-1) to (E′-3):

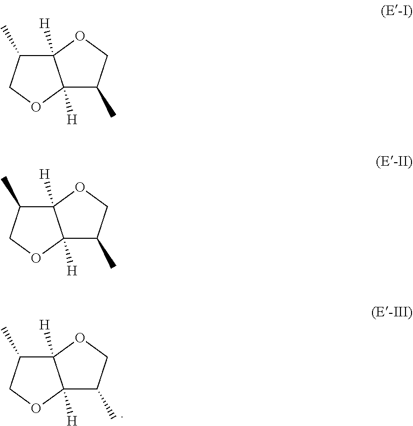

each R′ is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium; and
j′ is zero or an integer of 1 to 4;
is a bond or a divalent group optionally comprising one or more than one heteroatom; preferably T is selected from the group consisting of a bond, —CH₂—, —C(O)—, —C(CH₃)₂—, —C(CF₃)₂—, —C(═CCl₂)—, —C(CH₃)(CH₂CH₂COOH)—, and a group of formula:

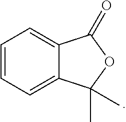

According to a preferred embodiment, the membrane (ME) comprises an amount of pore-forming agent of less than 0.1% wt., with respect to the overall weight of membrane (ME), for example of less than 0.09% wt. or less than 0.05% wt.

The Applicant has surprisingly found that polymers (PSI) are endowed with remarkable advantages over non bio-based aromatic sulfone polymers in the manufacture of filtration membranes. In particular, the Applicant observed that membranes (ME) obtained from polymer (PSI) are more hydrophilic and more antithrombogenic than membranes obtained from non bio-based aromatic sulfone polymers; as used herein, the term “antithrombogenic” means that the rate at which thrombosis occurs when whole blood is contacted with a membrane (M) is lower than that when whole blood is contacted with a membrane prepared starting from a composition free from the at least one polymer (F—PS). In addition, the Applicant observed that membranes (ME) comprising a polymer (PSI) and that do not contain pore-forming agents are more permeable to water than membranes obtained from non bio-based aromatic sulfone polymers.

This and other objects, advantages, and features of the invention will be more readily understood and appreciated by reference to the detailed description of the invention.

### DEFINITIONS

For the purposes of the present description:
the use of parentheses before and after symbols or numbers identifying compounds, chemical formulae or parts of formulae has the mere purpose of better distinguishing those symbols or numbers from the rest of the text and hence said parentheses can also be omitted;
unless otherwise indicated, the term “halogen” includes fluorine, chlorine, bromine or iodine and “halogenated” means containing one or more of fluorine, chlorine, bromine and iodine atoms;
the adjective “aromatic” denotes any mono- or polynuclear cyclic group (or moiety) having a number of π electrons equal to 4n°+2, wherein n° is 0 or any positive integer; an aromatic group (or moiety) can be an aryl or an arylene group (or moiety);
an “aryl group” is a hydrocarbon monovalent group consisting of one core composed of one benzenic ring or of a plurality of benzenic rings fused together by sharing two or more neighboring ring carbon atoms, and of one end. The end of an aryl group is a free electron of a carbon atom contained in a (or the) benzenic ring of the aryl group, wherein an hydrogen atom linked to said carbon atom has been removed. The end of an aryl group is capable of forming a linkage with another chemical group;
an “arylene group” is a hydrocarbon divalent group consisting of one core composed of one benzenic ring or of a plurality of benzenic rings fused together by sharing two or more neighboring ring carbon atoms, and of two ends. An end of an arylene group is a free electron of a carbon atom contained in a (or the) benzenic ring of the arylene group, wherein an hydrogen atom linked to said carbon atom has been removed. Each end of an arylene group is capable of forming a linkage with another chemical group;
when numerical ranges are indicated range ends are included;
a “biological fluid” is any fluid produced by a living organism, in particular by man, such as a blood product (including whole blood, plasma, or a fractionated blood component) urine, saliva and interstitial fluids.
   The polymer (PSI)

In polymer (PSI), the above recurring units of preferred embodiments (R_{PSI}-1) and (R_{PSI}-2) can be each present alone or in admixture.

More specifically, recurring units (R_{PSI}) of the polymer (PSI) are recurring units of any of formulae (R_{PSI}-1a), (R_{PSI}-1b), (R_{PSI}-1c), (R_{PSI}-2a), (R_{PSI}-2b), and (R_{PSI}-2c):

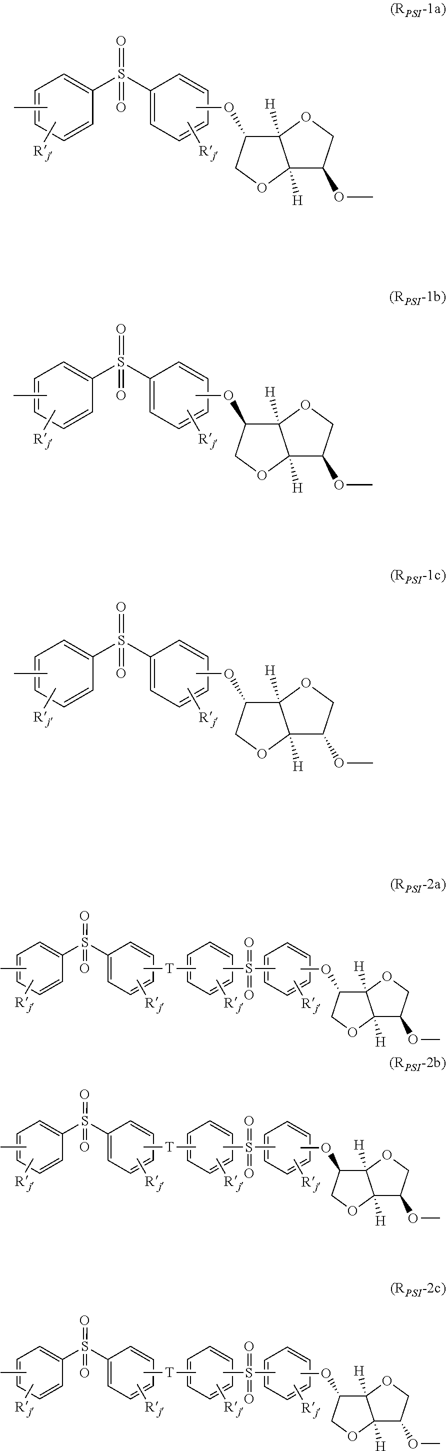

wherein
R′, J′ and T have the meaning as above defined.

The above recurring units of preferred embodiments (R_{PSI}-1a), (R_{PSI}-1 b), (R_{PSI}-1c), (R_{PSI}-2a), (R_{PSI}-2b), and (R_{PSI}-2c), can be each present alone or in admixture.

More preferred recurring units (R_{PSI}) are those of formula (R_{PSI}-1a) and (R_{PSI}-2a), optionally in combination with recurring units of formula (R_{PSI}-1b), (R_{PSI}-2b), (R_{PSI}-1c) and (R_{PSI}-2c).

Most preferred recurring units (R_{PSI}) are of formula (R_{PSI}-1a), optionally in combination with recurring units of formula (R_{PSI}-1b) and (R_{PSI}-1c).

In recurring unit (R_{PSI}), the respective phenylene moieties may independently have 1,2-, 1,4- or 1,3-linkages to the other moieties different from R′ in the recurring unit. Preferably, said phenylene moieties have 1,3- or 1,4-linkages, more preferably they have 1,4-linkage. Still, in recurring units (R_{PSI}) (including (R_{PSI}-1), (R_{PSI}-2), (R_{PSI}-1a), (R_{PSI}-1b), (R_{PSI}-1c), (R_{PSI}-2a), (R_{PSI}-2b), and (R_{PSI}-2c)), j′ is at each occurrence zero, that is to say that the phenylene moieties have no other substituents than those enabling linkage in the main chain of the polymer.

Polymer (PSI) may comprise, in addition to recurring units (R_{PSI}), as detailed above, recurring units (R_{S}) comprising a Ar—SO₂—Ar′ group, with Ar and Ar′, equal to or different from each other, being aromatic groups, said recurring units (Rₛ) generally complying with formulae (S1)

—Ar⁵-(T′-Ar⁶)ₙ—O—Ar⁷—SO₂—[Ar⁸-(T-Ar⁹)ₙ—SO₂]ₘ—Ar¹⁰—O— (S1):

wherein:
Ar⁵, Ar⁶, Ar⁷, Ar⁸, and Ar⁹, equal to or different from each other and at each occurrence, are independently an aromatic mono- or polynuclear group;
T and T′, equal to or different from each other and at each occurrence, is independently a bond or a divalent group optionally comprising one or more than one heteroatom; preferably T and T′ are selected from the group consisting of a bond, —CH₂—, —C(O)—, —C(CH₃)₂—, —C(CF₃)₂—, —C(═CCl₂)—, —C(CH₃)(CH₂CH₂COOH)—, —SO₂— and a group of formula:

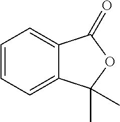

most preferably, T′ is a bond, —SO₂—, or —C(CH₃)₂— and T is a bond;
n and m, equal to or different from each other, are independently zero or an integer of 1 to 5.

Recurring units (R_{S}) can be notably selected from the group consisting of those of formulae (S-A) to (S-D) herein below:

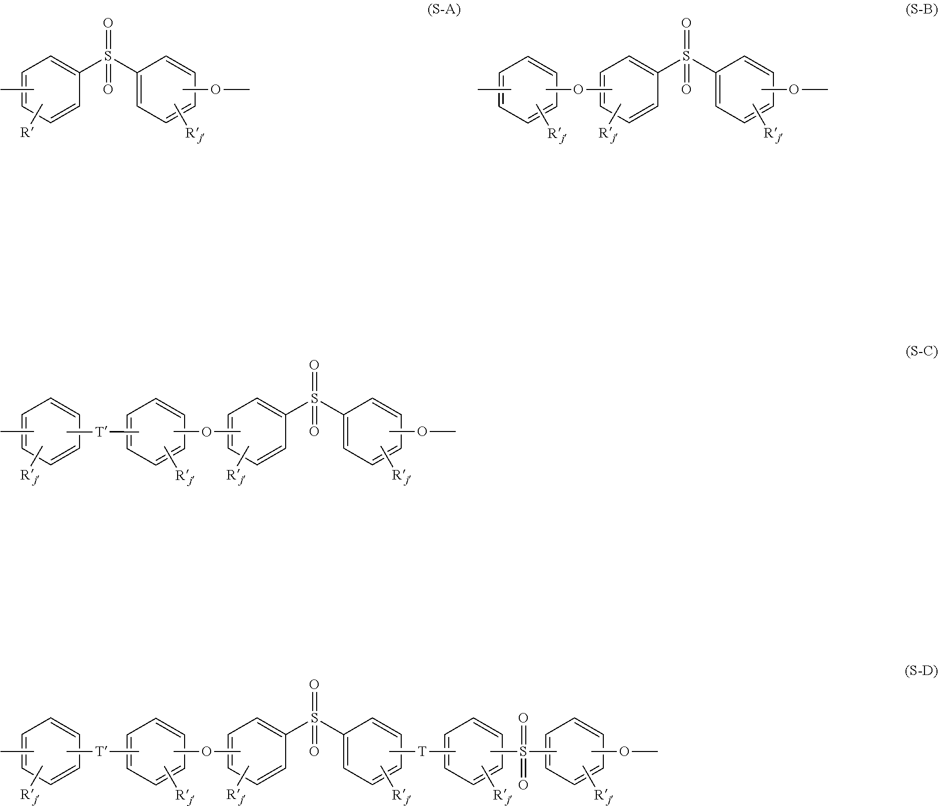

wherein:
each of R′, equal to or different from each other, is selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium;
j′ is zero or is an integer from 0 to 4;
T and T′, equal to or different from each other are a bond or a divalent group optionally comprising one or more than one heteroatom; preferably T and T′ are selected from the group consisting of a bond, —CH₂—, —C(O)—, —C(CH₃)₂—, —C(CF₃)₂—, —C(═CCl₂)—, —C(CH₃) (CH₂CH₂COOH)—, —SO₂—, and a group of formula:

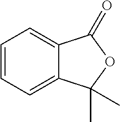

most preferably, T′ is a bond, —SO₂—, or—C(CH₃)₂— and T is a bond. In recurring unit (R_{S}), the respective phenylene moieties may independently have 1,2-, 1,4- or 1,3-linkages to the other moieties different from R′ in the recurring unit. Preferably, said phenylene moieties have 1,3- or 1,4-linkages, more preferably they have 1,4-linkage. Still, in recurring units (R_{S}), j′ is at each occurrence zero, that is to say that the phenylene moieties have no other substituents than those enabling linkage in the main chain of the polymer.

Recurring units (R_{S}) of formula (S-D) are preferably selected from the group consisting of the following recurring units:

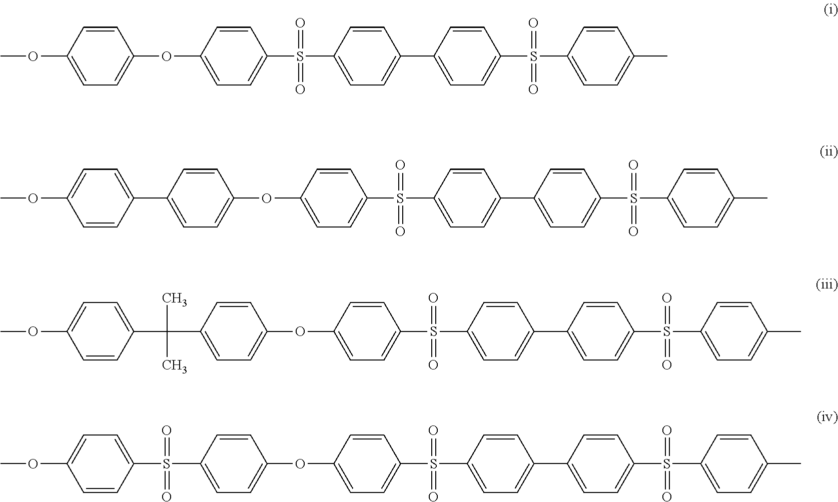

and mixtures thereof.

Recurring units (R_{S}) complying with formula (S—C), as above detailed, are preferably selected from the group consisting of the following units:

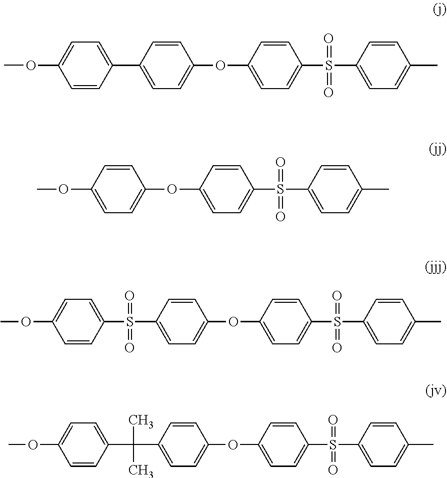

and mixtures thereof.

The polymer (PSI) has in general a weight average molecular weight of at least 20 000 preferablv at least 30 000, more referablv at least 40 000.

The weight average molecular weight (M_{w}) and the number average molecular weight (Mₙ) can be estimated by gel-permeation chromatography (GPC) using ASTM D5296 calibrated with polystyrene standards.

The polydispersity index (PDI) is hereby expressed as the ratio of weight average molecular weight (M_{w}) to number average molecular weight (Mₙ).

The polymer (PSI) generally has a polydispersity index of less than 2.5, preferably of less than 2.4, more preferably of less than 2.2. This relatively narrow molecular weight distribution is representative of an ensemble of molecular chains with similar molecular weights and substantially free from oligomeric fractions, which might have a detrimental effect on polymer properties.

The polymer (PSI) advantageously possesses a glass transition temperature (T_{g}) of at least 200° C., preferably 210° C., more preferably at least 220° C. Such high glass transition temperatures are advantageous for extending temperatures range of use of the polymer (PSI).

Glass transition temperature (T_{g}) is generally determined by DSC, according to ASTM D3418.

The polymer (PSI) comprises recurring units (R_{PSI}), as above detailed, in an amount of more than 50% moles, preferably more than 60% moles, more preferably more than 75% moles, even more preferably more than 80% moles, with respect to all the recurring units of polymer (PSI).

When recurring units different from units (R_{PSI}) are present in polymer (PSI), the same are generally selected from recurring units (R_{S}), as above detailed, so that polymer (PSI) essentially consists of recurring units (R_{PSI}), as above detailed, and, optionally, recurring units (R_{S}), as above detailed.

End chains, defects, and minor amounts (<1% moles, with respect to all the recurring units of polymer (PSI)) of recurring units other than recurring units (R_{PSI}), and recurring units (R_{S}), may be present, without this presence substantially affecting the properties of the polymer (PSI).

It is generally understood that good results can be achieved using a polymer (PSI) wherein substantially all recurring units are recurring units (R_{PSI}), as above detailed.

The expression “substantially” in combination with the recited amount of recurring units (R_{PSI}) is hereby intended to mean that minor amounts, generally below 1% moles, preferably below 0.5% moles, of other recurring units may be tolerated, e.g. as a result of lower purity in monomers used.

### Purification Methods [Methods (MPUR)] and Membranes (ME)

As stated above, purification methods (MPUR) according to the present invention comprise at least one filtration step of a biological fluid through a membrane (ME), said membrane (ME) being obtained from a polymer (PSI).

Preferably, purification methods (MPUR) are methods for purifying a human biological fluid, preferably a blood product, such as whole blood, plasma, fractionated blood components or mixtures thereof, that are carried out in an extracorporeal circuit. The extracorporeal circuit for carrying out a method (MPUR) comprises at least one filtering device (or filter) comprising at least one membrane (ME).

As intended herein, a blood purification method through an extracorporeal circuit comprises hemodyalisis (FD) by diffusion, hemofiltration (HF), hemodyafiiltration (HDF) and hemoconcentration. In HF, blood is filtered by ultrafiltration, while in HDF blood is filtered by a combination of FD and HF.

Blood purification methods (MPUR) through an extracorporeal circuit are typically carried out by means of a hemodyalizer, i.e. an equipment designed to implement any one of FD, HF or HFD. In such methods, blood is filtered from waste solutes and fluids, like urea, potassium, creatinine and uric acid, thereby providing waste solutes- and fluids-free blood.

Therefore, in one aspect, the present invention relates to a hemodyalizer comprising at least one membrane (ME).

Typically, a hemodyalizer for carrying out a blood purification method (MPUR) comprises a cylindrical bundle of hollow fibers of membranes (ME), said bundle having two ends, each of them being anchored into a so-called potting compound, which is usually a polymeric material acting as a glue which keeps the bundle ends together. Potting compounds are known in the art and include notably polyurethanes; convenient examples of potting compounds are cited in US 2011/0009799. The potted cylindrical bundle is put into a clear plastic cyclindrical shell with four openings (or blood ports). Two of such openings are at the ends of the cyclindrical shell and are in communication with the each end of the bundle of hollow fibers, thereby forming the “blood compartment” of the dialyzer, while the other two openings are cut into the side of the cylinder and communicate with the so called “dialysate compartment” of the dialyzer. By applying a pressure gradient, blood is pumped through the bundle of membranes (ME) via the blood ports and the filtration product (the “dialysate”) is pumped through the space surrounding the filers.

The term “membrane” is used herein in its usual meaning, that is to say it refers to a discrete, generally thin, interface that moderates the permeation of chemical species in contact with it. This interface may be molecularly homogeneous, that is, completely uniform in structure (dense membrane), or it may be chemically or physically heterogeneous, for example containing voids, holes or pores of finite dimensions (porous membrane).

Membrane (ME) is typically a microporous membrane which can be generally characterized by its average pore diameter and porosity, i.e. the fraction of the total membrane that is porous. Membrane (ME) has a gravimetric porosity (εₘ) of 20 to 90% and comprises pores, wherein at least 90% by volume of the said pores has an average pore diameter of less than 5 μm.

Membranes having a uniform structure throughout their thickness are generally known as symmetrical membranes; membranes having pores which are not homogeneously distributed throughout their thickness are generally known as asymmetric membranes. Asymmetric membranes are characterized by a thin selective layer (0.1-1 μm thick) and a highly porous thick layer (100-200 μm thick) which acts as a support and has little effect on the separation characteristics of the membrane.

Membranes (ME) can be in the form of a flat sheet or in the form of tubes. Tubular membranes are classified based on their dimensions in tubular membranes having a diameter greater than 3 mm; capillary membranes, having a diameter comprised between 0.5 mm and 3 mm; and hollow fibers having a diameter of less than 0.5 mm. Capillary membranes are otherwise referred to as hollow fibres.

Hollow fibres are particularly advantageous in applications where compact modules with high surface areas are required. Hollow fibres membranes are preferred when method (MPUR) is a method for the filtration of blood through an extracorporeal circuit, preferably through a hemodialyzer.

Membranes (ME) may also be supported to improve their mechanical resistance. The support material is selected to have a minimal influence on the selectivity of the membrane.

Typically, membranes (ME) suitable for carrying out method (MPUR) of the invention have an asymmetric structure.

The gravimetric porosity of membranes (ME) may range from 20 to 90%, preferably from 30 to 80%.

As explained, the average pores diameter (also referred to as “voids”) can be measured taking SEM picture from surfaces of fractured sections of microporous membranes (ME). Fractured sections are obtained fracturing a membrane (ME) in liquid nitrogen in a parallel direction to the intended direction of flow through the membrane; fracturing in the said conditions is efficient in ensuring geometry and morphology to be preserved and avoiding any ductile deformation.

Manual or automated analysis of SEM pictures taken at suitable magnification/resolution enables delivering data regarding the average pores diameter.

The expression “average diameter” is meant to indicate that for pore sections of non-spherical shape, an average diameter is computed considering the average between the longest axis and the shortest axis perpendicular thereto, while for spherical shapes, the actual geometrical diameter is to be taken as average diameter.

The pores may have an average diameter of at least 0.001 μm, of at least 0.005 μm, of at least 0.01 μm. The pores may have an average diameter of at most 5 μm, preferably at most 4 μm, even more preferably at most 3 μm.

Microporous membranes (ME) for carrying out method (MPUR) of the invention generally possesses a water flux permeability, at a pressure of 1 bar and at a temperature of 23° C., of at least 300, preferably at least 400, more preferably at least 500 l/(h×m²).

Membranes (ME) according to the present invention can be manufactured according to methods known in the art. Preferably, membranes (ME) are prepared by a phase inversion method occurring in the liquid phase, said method [method (MM-1)] comprising the following steps:
(i) preparing a polyaryl ether sulfone polymer solution [solution (SP)] comprising a sulfone polymer (PSI) above described and a polar solvent [solvent (S)];
(ii) processing said solution (SP) into a film;
(iii) contacting said film with a non-solvent bath.

Solvent (S) is typically a polar organic solvent.

The term “solvent” is used herein in its usual meaning, that is it indicates a substance capable of dissolving another substance (solute) to form an uniformly dispersed mixture at the molecular level. In the case of a polymeric solute it is common practice to refer to a solution of the polymer in a solvent when the resulting mixture is transparent and no phase separation is visible in the system. Phase separation is taken to be the point, often referred to as “cloud point”, at which the solution becomes turbid or cloudy due to the formation of polymer aggregates.

Exemplary solvents (S) which may be used, alone or in combination, to prepare a solution (SP):
aromatic hydrocarbons and more particularly aromatic hydrocarbons such as, in particular, benzene, toluene, xylenes, cumene, petroleum fractions composed of a mixture of alkylbenzenes:
aliphatic or aromatic halogenated hydrocarbons including more particularly, perchlorinated hydrocarbons such as, in particular, tetrachloroethylene, hexachloroethane; partially chlorinated hydrocarbons such as dichloromethane, chloroform, 1,2-dichloroethane, 1,1,2-trichloroethane, 1,1,2,2-tetrachloroethane, pentachloroethane, trichloroethylene, 1-chlorobutane, 1,2-dichlorobutane; monochlorobenzene, 1,2-dichlorobenzene, 1,3-dichlorobenzene, 1,4-dichlorobenzene, 1,2,4-trichlorobenzene or mixture of different chlorobenzenes;
aliphatic, cycloaliphatic or aromatic ether oxides, more particularly, diethyl oxide, dipropyl oxide, diisopropyl oxide, dibutyl oxide, methyltertiobutylether, dipentyl oxide, diisopentyl oxide, ethylene glycol dimethyl ether, ethylene glycol diethyl ether, ethylene glycol dibutyl ether benzyl oxide; 1,4-dioxane, tetrahydrofuran (THF);
aromatic amines, including notably pyridine, and aniline.
ketones such as methylethylketone, methylisobutyl ketone, diisobutylketone, cyclohexanone, isophorone;
linear or cyclic esters such as: isopropyl acetate, n-butyl acetate, methyl acetoacetate, dimethyl phthalate, γ-butyrolactone;
linear or cyclic carboxamides such as N,N-dimethylacetamide (DMAc), N,N-diethylacetamide, dimethylformamide (DMF), diethylformamide or N-methyl-2-pyrrolidinone (NMP);
organic carbonates for example dimethyl carbonate, diethyl carbonate, dipropyl carbonate, dibutyl carbonate, ethylmethyl carbonate, ethylene carbonate, vinylene carbonate;
phosphoric esters such as trimethyl phosphate, triethyl phosphate;
dimethylsulfoxide (DMSO); and
diesters of formula (I_{de}), ester-amides of formula (Iₑₐ), or diamides of formula (I_{da}):

R¹—OOC-A_{de}-COO—R² (I_{de})

R¹—OOC-Aₑₐ-CO—NR³R⁴ (Iₑₐ)

R⁵R⁶N—OC-A_{da}-CO—NR⁵R⁶ (I_{da})

wherein:
R¹ and R², equal to or different from each other, are independently selected from the group consisting of C₁-C₂₀ hydrocarbon groups;
R³, R⁴, R⁵ and R⁶ equal to or different from each other and at each occurrence, are independently selected from the group consisting of hydrogen, C₁-C₃₆ hydrocarbon groups, possibly substituted, being understood that R³ and R⁴ might be part of a cyclic moiety including the nitrogen atom to which they are bound, said cyclic moiety being possibly substituted and/or possibly comprising one or more than one additional heteroatom, and mixtures thereof;
A_{de}, Aₑₐ, and A_{da} equal to or different from each other, are independently a linear or branched divalent alkylene group.

In one embodiment, solvent (S) is at least one of the group consisting of NMP, DMAc, pyridine, aniline, 1,1,2-trichloroethane and 1,1,2,2-tetrachloroethane, tetrahydrofuran (THF), 1,4 dioxane, chloroform, dichloromethane, and chlorobenzene.

Very good results have been obtained when the solvent (S) was NMP or DMAc.

In another embodiment, solvent (S) is at least one of a diester of formula (I_{de}), or an ester-amide of formula (Iₑₐ), possibly in admixture with a diamides of formula (I_{da}), wherein A in formulae (I_{de}), (Iₑₐ) and (I_{da}) is C₃-C₁₀ branched divalent alkylene.

According to this embodiment, A is preferably selected from the group consisting of the following:
A_{MG} groups of formula MGₐ-CH(CH₃)—CH₂—CH₂— or MG_{b}-CH₂—CH₂—CH(CH₃)—,
A_{ES} groups of formula ESₐ—CH(C₂H₅)—CH₂—, or ES_{b}—CH₂—CH(C₂H₅)—; and
mixtures thereof.

In one more preferred variant of this embodiment, the solvent (S) comprises, possibly in addition to DMSO:
(i) at least one of the diester (I′_{de}) and at least one diester (I″_{de}), possibly in combination with at least one diester of formula (II_{de}); or
(ii) at least one of the esteramide (I′ₑₐ) and at least one esteramide (I″ₑₐ), possibly in combination with at least one esteramide of formula (IIₑₐ);
(iii) at least one of the esteramide (I′ₑₐ), at least one diamide (I′_{da}), at least one esteramide (I″ₑₐ) and at least one diamide (I″_{da}), possibly in combination with at least one esteramide of formula (ilea) and/or at least one diamide of formula (II_{da}); or
(iv) combinations of (i) with (ii) and/or (iii),

wherein:
(I′_{de}) is R¹—OOC-A_{MG}-COO—R²
(I′ₑₐ) is R¹—OOC-A_{MG}-CO—NR³R⁴
(I′_{da}) is R⁵R⁶N—OC-A_{MG}-CO—NR⁵R⁶
(I″_{de}) is R¹—OOC-A_{ES}-COO—R²
(I″ₑₐ) is R⁵R⁶N—OC-A_{ES}-CO—NR⁵R⁶; and
(II_{de}) is R¹—OOC—(CH₂)₄—COO—R²,
(ilea) is R¹—OOC—(CH₂)₄—CO—NR³R⁴,
(II_{da}) is R⁵R⁶N—OC—(CH₂)₄—CO—NR⁵R⁶,

wherein:
A_{MG} is of formula MGₐ-CH(CH₃)—CH₂—CH₂— or MG_{b}-CH₂—CH₂—CH(CH₃)—,
A_{ES} is of formula ESₐ—CH(C₂H₅)—CH₂—, or ES_{b}—CH₂—CH(C₂H₅)—; and wherein R¹ and R², equal to or different from each other, are independently selected from the group consisting of C₁-C₂₀ alkyl, C₁-C₂₀ aryl, C₁-C₂₀ alkyaryl, C₁-C₂₀ arylalkyl groups;
R³, R⁴, R⁵ and R⁶, equal to or different from each other and at each occurrence, are selected from the group consisting of C₁-C₂₀ alkyl, C₁-C₂₀ aryl, C₁-C₂₀ alkyaryl, C₁-C₂₀ arylalkyl groups, all said groups possibly comprising one or more than one substituent, possibly having one or more than one heteroatom, and of cyclic moieties comprising both (1) R³ and R⁴ or R⁵ and R⁶ and (2) the nitrogen atom to which they are bound, said cyclic moieties possibly comprising one or more than one heteroatom, e.g. an oxygen atom or an additional nitrogen atom.

In above mentioned formulae (I′_{de}), (I″_{de}), and (II_{de}), (I′ₑₐ), (I″ₑₐ) and (IIₑₐ), (I′_{da}), (I″_{da}) and (II_{da}), R¹ and R² are preferably methyl groups, while R³, R⁴, R⁵ and R⁶ equal to or different from each other and at each occurrence, are preferably selected from the group consisting of methyl, ethyl, hydroxyethyl.

In this preferred variant of this embodiment, the solvent (S) preferably consists essentially of any of (i), (ii), (iii) or (iv) mixtures, possibly in combination with DMSO. Other minor components might be present, preferably in an amount not exceeding 1% wt over the entire weight of the solvent (S), provided they do not substantially modify the properties of solvent (S).

According to this variant, solvent (S) can comprise (or consist essentially of), possibly in addition to DMSO:
(j) a diester mixture consisting essentially of:
   from 70 to 95% by weight of diester of formula (I′_{de});
   from 5 to 30% by weight of diester of formula (I″_{de}), and
   from 0 to 10% by weight of diester of formula (II_{de}), as above detailed; or
(jj) an esteramide mixture consisting essentially of:
   from 70 to 95% by weight of esteramide of formula (I′ₑₐ);
   from 5 to 30% by weight of esteramide of formula (I″ₑₐ), and
   from 0 to 10% by weight of any of esteramide of formula (Ilea), as above detailed; or
(jjj) an esteramide/diamide mixture consisting essentially of
   from 70 to 95% by weight of esteramide of formula (I′ₑₐ) and diamide of formula (I′_{da}), with (I′_{da}) representing from 0.01 to 10% by weight of cumulative weigh of (I′ₑₐ) and (I′_{da});
   from 5 to 30% by weight of esteramide of formula (I″ₑₐ) and diamide of formula (I″_{da}), with (I″_{da}) representing from 0.01 to 10% by weight of cumulative weigh of (I″ₑₐ) and (I″_{da}) and
   from 0 to 10% by weight of any of esteramide of formule (IIₑₐ) and diamide (II_{da}), as above detailed; or

mixtures of (j) with (jj) and/or (jjj) as above detailed.

An example of useful esteramide-based mixture is RHODIASOLV® PolarClean, comprising essentially methyl 5-(dimethylamino)-2-methyl-5-oxopentanoate.

In one other embodiment, solvent (S) is at least one of a diester of formula (I_{de}), or an ester-amides of formula (Iₑₐ), possibly in admixture with a diamides of formula (I_{da}), wherein A in formulae (I_{de}), (Iₑₐ) and (I_{da}) is a linear divalent alkylene group of formula (CH₂)ᵣ, wherein r is an integer of from 2 to 4.

In a variant of this embodiment, the solvent (S) comprises, possibly in addition to DMSO:
(k) at least one of the diester of formula (III⁴_{de}), the diester of formula (III³_{de}), and the diester of formula (III²_{de}); or
(kk) at least one of the esteramide (III⁴ₑₐ), the esteramide (III³ₑₐ), and the esteramide of formula (III²ₑₐ); or
(kkk) at least one of the esteramide of formula (III⁴ₑₐ), the esteramide of formula (III³ₑₐ), and the esteramide of formula (III²ₑₐ), and at least one of the diamide of formula (III⁴_{da}), the diamide of formula (III³_{da}), and the diamidee of formula (III²_{da}); or
(kv) combinations of (k) with (kk) and/or (kkk),
wherein:
   (III⁴_{de}) is R¹—OOC—(CH₂)₄—COO—R²
   (III³_{de}) is R¹—OOC—(CH₂)₃—COO—R²
   (III²_{de}) is R¹—OOC—(CH₂)₂—COO—R²
   (II⁴ₑₐ) is R¹—OOC—(CH₂)₄—CO—NR³R⁴
   (II³ₑₐ) is R¹—OOC—(CH₂)₃—CO—NR³R⁴
   (III²ₑₐ) is R¹—OOC—(CH₂)₂—CO—NR³R⁴
   (III⁴_{da}) is R⁵R⁶N—OC—(CH₂)₄—CO—NR⁵R⁶
   (III³_{da}) is R⁵R⁶N—OC—(CH₂)₃—CO—NR⁵R⁶
   (III²_{da}) is R⁵R⁶N—OC—(CH₂)₂—CO—NR⁵R⁶
wherein R¹ and R², equal to or different from each other, are independently C₁-C₂₀ alkyl, C₁-C₂₀ aryl, C₁-C₂₀ alkyaryl, C₁-C₂₀ arylalkyl groups;
   R³, R⁴, R⁵ and R⁶, equal to or different from each other and at each occurrence, are selected from the group consisting of C₁-C₂₀ alkyl, C₁₀C₂₀ aryl, C₁-C₂₀ alkyaryl, C₁-C₂₀ arylalkyl groups, all said groups possibly comprising one or more than one substituent, possibly having one or more than one heteroatom, and of cyclic moieties comprising both (1) R³ and R⁴ or R⁵ and R⁶ and (2) the nitrogen atom to which they are bound, said cyclic moieties possibly comprising one or more than one heteroatom, e.g. an oxygen atom or an additional nitrogen atom.

In above mentioned formulae (III⁴_{de}), (III³_{de}), (III²_{de}), (III⁴ₑₐ), (III³ₑₐ), and (III²ₑₐ), (III⁴_{da}), (III³_{da}), and (III²_{da}), R¹ and R² are preferably methyl groups, while R³, R⁴, R⁵ and R⁶, equal to or different from each other, are preferably selected from the group consisting of methyl, ethyl, hydroxyethyl.

According to certain preferred variant of this embodiment, solvent (S) can comprise, possibly in addition to DMSO:
(I) a diester mixture consisting essentially of dimethyladipate (r=4), dimethylglutarate (r=3) and dimethylsuccinate (r=2); or
(II) an esteramide mixture consisting essentially of H₃COOC—(CH₂)₄—CO—N(CH₃)₂, H₃COOC—(CH₂)₃—CO—N(CH₃)₂, and H₃COOC—(CH₂)₂—CO—N(CH₃)₂; or
(III) a diester mixture of diethyladipate (r=4), diethylglutarate (r=3) and diethylsuccinate (r=2); or
(Iv) an esteramide mixture consisting essentially of H₅C₂OOC—(CH₂)₄—CO—N(CH₃)₂, H₅C₂OOC—(CH₂)₃—CO—N(CH₃)₂, and H₅C₂OOC—(CH₂)₂—CO—N(CH₃)₂; or
(v) a mixture of diisobutyladipate (r=4), diisobutylglutarate (r=3) and diisobutylsuccinate (r=2); or
(vI) an esteramide mixture consisting essentially of H₉C₄OOC—(CH₂)₄—CO—N(CH₃)₂, H₉C₄OOC—(CH₂)₃—CO—N(CH₃)₂, and H₉C₄OOC—(CH₂)₂—CO—N(CH₃)₂; or
(vII) mixtures thereof.

An exemplary embodiment of the variant listed above under section (I) is a diester mixture consisting essentially of:
from 9 to 17% by weight of dimethyladipate;
from 59 to 67% by weight of dimethylglutarate; and
from 20 to 28% by weight of dimethylsuccinate.

An example of a useful diester-based mixture wherein A is linear is RHODIASOLV® RPDE solvent, marketed by Solvay.

RHODIASOLV® RPDE solvent is a mixture of diesters comprising essentially (more than 70 wt %) of dimethylglutarate and dimethylsuccinate.

According to certain other embodiments, solvent (S) comprises dimethylsulfoxide (DMSO) and at least one solvent selected from the group consisting of diesters of formula (I_{de}) and ester-amide of formula (Iₑₐ).

The weight ratio between the solvents of formula (I_{de}) and (Iₑₐ) and DMSO, in these embodiments, is preferably from 1/99 to 99/1, preferably of from 20/80 to 80/20, more preferably of 70/30 to 30/70. The skilled in the art will select the appropriate weight ratio for opportunely tuning properties of the solvent (S) in the inventive composition.

The overall concentration of the solvent (S) in the solution (SP) should be at least 20% by weight, preferably at least 30% by weight, based on the total weight of the solution. Typically the concentration of the solvent (S) in the solution does not exceed 70% by weight, preferably it does not exceed 65% by weight, more preferably it does not exceed 60% by weight, based on the total weight of the solution (SP).

The solution (SP) may contain additional components, such as nucleating agents, fillers and the like.

According to an embodiment of the present invention, the membrane is free from pore forming agent [agent (A)].

Examples of pore forming agents are notably polyvinylpyrrolidone (PVP), and polyethyleneglycol (PEG) having a molecular weight of at least 200.

According to another embodiment, The pore forming agent, when added to the solution (SP), it is present in amounts typically ranging from 0.1 to 40% by weight, preferably from 0.5 to 40% by weight.

When PEG pore forming agents are used, their amounts is generally of from 30 to 40% wt, with respect to the total weight of solution (SP); when PVP pore forming agents are employed, their amounts is generally of 2 to 10% wt, with respect to the total weight of solution (SP).

Particularly good results have been obtained with solutions (SP) wherein the agent (A) is a polyvinylpirrolidone (PVP), as above detailed. However, the Applicant observed that, even if the pore-forming agent is removed, in whole or in part, from membrane (ME), the permeability to water and the wettability of the membranes remain higher than those of membranes comprising aromatic sulfone polymers not based on biological stocks.

The overall concentration of the polymer (PSI) in the solution (SP) should be at least 8% by weight, preferably at least 12% by weight, based on the total weight of the solution. Typically the concentration of the polymer (PSI) in the solution does not exceed 50% by weight, preferably it does not exceed 40% by weight, more preferably it does not exceed 30% by weight, based on the total weight of the solution (SP).

The concentration of polymer (PSI) ranging from 15 to 25% wt with respect to the total weight of solution (SP) have been found particularly advantageous.

The solution (SP) can be prepared in step (i) by any conventional manner. For instance, the solvent (S) can be added to the polymer (PSI), followed by mixture (PHA), and possibly agent (A), or, preferably, the polymer (PSI) can be admixed with agent (A) and mixture (PHA) before being contacted with the solvent (S). No specific effects can be associated to the order of contacting combining the ingredients.

Step (i) is generally carried out at a temperature of advantageously at least 25° C., preferably at least 30° C., more preferably at least 40° C. and even more preferably at least 45° C. Step (i) is generally carried out at a temperature of advantageously less than 180° C., preferably less than 170° C., more preferably less than 160° C., and even more preferably less than 150° C. Higher temperatures can of course be used for the solution (SP) preparation step (i), however they are not preferred from a practical and/or economical point of view.

The mixing time required to obtain the solution (SP) can vary widely depending upon the rate of solution of the components, the temperature, the efficiency of the mixing apparatus, the viscosity of the solution (SP) being prepared, and the like.

Any suitable mixing equipment may be used. Preferably, the mixing equipment is selected to reduce the amount of air entrapped in the solution (SP) which may cause defects in the final membrane. The mixing of the polymer (P) and the solvent (S) and the mixture (PHA) may be conveniently carried out in a sealed container, optionally held under an inert atmosphere. Inert atmosphere, and more precisely nitrogen atmosphere has been found particularly advantageous for the preparation of solution (SP).

In general the solubility of the polymer (PSI) in the solution (SP) at the temperature of the solution during the step (ii) of the method of the invention should be greater than 10% by weight, preferably greater than 12% by weight, more preferably greater than 15% by weight, with respect to the total weight of the solution (SP).

The term “solubility” is defined herein as the maximum amount of polymer, measured in terms of weight of the polymer per weight of solution, which dissolves at a given temperature affording a transparent homogeneous solution without the presence of any phase separation in the system.

For this reason, step (ii) may be carried out at temperatures exceeding room temperature. Once a homogenous and transparent solution (SP) is prepared, the solution (SP) is processed into a film.

The term “film” is used herein to refer to the layer of solution (SP) obtained after the processing of the same. Depending on the final form of the membrane the film may be either flat, when flat membranes are to be manufactured, or tubular in shape, when tubular or hollow fiber membranes are to be obtained.

The temperature during the processing step (ii) may be or may be not the same as the temperature during the preparation step (i). The temperature of the solution (SP) during the processing step (ii) typically does not exceed 180° C., preferably it does not exceed 170° C., more preferably it does not exceed 160° C., even more preferably it does not exceed 150° C.

During the processing step (ii) the solution (SP), lower boundary for the processing temperature are not critical, provided that the solution (SP) still maintains adequate solubility and viscosity properties. Ambient temperature can be notably used.

From practical perspective, nevertheless, the temperature of the solution (SP) during the processing step (ii) generally is comprised between 30° C. and 70° C., preferably between 30° C. and 50° C.

The viscosity of the solution (SP) at the temperature of the processing step (ii) is typically at least 1 Pa·s. The viscosity of the solution (SP) in said conditions typically does not exceed 100 Pa·s. This viscosity window can be adapted adjusting notably polymer (PSI), mixture (PHA), agent (A) and solvent (S) relative proportions in the solution (SP), and through additional adjustment of the temperature, as mentioned above.

Conventional techniques can be used for processing the solution (SP) into a film, including casting and wet-spinning.

Different casting techniques can be used depending on the final form of membrane (ME). When membrane (ME) is a flat membrane, solution (S) is cast as a film over a flat support, typically a plate, a belt or a fabric, or another microporous supporting membrane, by means of a casting knife or a draw-down bar.

Accordingly, in one embodiment, method (MM) comprises a step (ii) of casting the solution (SP) into a flat film on a support.

Hollow fibers and capillary membranes (ME) can be obtained by the so-called wet-spinning process. In such a process, the solution (SP) is generally pumped through a spinneret, that is an annular nozzle comprising at least two concentric capillaries: a first outer capillary for the passage of the solution (SP) and a second inner one for the passage of a supporting fluid, generally referred to as “lumen”. The lumen acts as the support for the casting of the solution (SP) and maintains the bore of the hollow fiber or capillary precursor open. The lumen may be a gas, or, preferably, a liquid at the conditions of the spinning of the fiber. The selection of the lumen and its temperature depends on the required characteristics of the final membrane as they may have a significant effect on the size and distribution of the pores in the membrane. In general the lumen is not a strong non-solvent for the polymer (PSI) or, alternatively, it contains a solvent or weak solvent for the polymer (PSI). The lumen is typically miscible with the non-solvent and with the solvent (S) for the polymer (PSI). The temperature of the lumen generally approximates the temperature of the solution (SP).

At the exit of the spinneret, after a short residence time in air or in a controlled atmosphere, the hollow fiber or capillary precursor is contacted with a non-solvent, and more specifically it is generally immersed in the non-solvent bath wherein the polymer precipitates forming the hollow fiber or capillary membrane.

Accordingly, in a second embodiment, method (MM) comprises a step (ii) of casting the polymer solution into a tubular film around a supporting fluid. The casting of the polymer solution is typically done through a spinneret. The supporting fluid forms the bore of the final hollow fiber or capillary membrane. When the supporting fluid is a liquid, immersion of the fiber precursor in the non-solvent bath also advantageously removes the supporting fluid from the interior of the fiber.

According to this embodiment, the supporting fluid is generally selected from non-solvents for the polymer (PSI), and more specifically from water and aliphatic alcohols, preferably, aliphatic alcohols having a short chain, for example from 1 to 6 carbon atoms, more preferably methanol, ethanol and isopropanol, and mixtures comprising the same.

Blends of said preferred non-solvents, i.e. comprising water and one or more aliphatic alcohols can be used.

Preferably, the supporting fluid is selected from the group consisting of
water,
aliphatic alcohols as above defined, and mixture thereof.

Most preferably, the supporting fluid is water.

Tubular membranes (ME), because of their larger diameter, are produced using a different method (MM) from the one employed for the production of hollow fiber membranes. For this purpose, a method (MM) comprises a step (ii) of casting the polymer solution into a tubular film over a supporting tubular material.

After the processing of the solution (SP) has been completed so as to obtain a film, in whichever form, as above detailed, said film is contacted with a non-solvent bath in step (iii). This step is generally effective for inducing the precipitation of the polymer (PSI) from the solution (SP). The precipitated polymer (PSI) thus advantageously forms the final membrane structure.

As used herein the term “non-solvent” is taken to indicate a substance incapable of dissolving a given component of a solution or mixture.

Suitable non-solvents for the polymer (PSI) are water and aliphatic alcohols, preferably, aliphatic alcohols having a short chain, for example from 1 to 6 carbon atoms, more preferably methanol, ethanol and isopropanol. Blends of said preferred non-solvents, i.e. comprising water and one or more aliphatic alcohols can be used. Preferably, the non-solvent of the non-solvent bath is selected from the group consisting of—water,
aliphatic alcohols as above defined, and mixture thereof. Further in addition, the non-solvent bath may comprise in addition to the non-solvent (e.g. in addition to water, to aliphatic alcohol or to mixture of water and aliphatic alcohols, as above detailed) small amounts (typically of up to 40% wt, with respect to the total weight of the non-solvent bath, generally 25 to 40% wt)) of a solvent for the polymer (PSI). Use of solvent/non-solvent mixtures advantageously allows controlling the porosity of the membrane. The non-solvent is generally selected among those miscible with the solvent (S) used for the preparation of the solution (SP). Preferably the non-solvent in method (MM) is water. Water is the most inexpensive non-solvent and it can be used in large amounts. The solvent (S) is advantageously selected so as to be miscible and soluble in water, which is an additional advantage of the method of the present invention.

The non-solvent in the precipitation bath is usually held at a temperature of at least 0° C., preferably of at least 15° C., more preferably of at least 20° C. The non-solvent in the precipitation bath is usually held at a temperature of less than 90° C., preferably of less than 70° C., more preferably of less than 60° C.

The temperature gradient between the cast film and the non-solvent bath may influence the pore size and/or pore distribution in the final membrane as it affects the rate of precipitation of the polymer (PSI) from the solution (SP). If precipitation is rapid, a skin will generally form on the surface of the cast film in contact with the non-solvent which will typically slow down the diffusion of the non-solvent in the bulk of the polymer solution leading to a membrane with an asymmetric structure. If precipitation is slow, the pore-forming liquid droplets of the solvent-rich liquid phase, which forms upon contact with the non-solvent, usually tend to agglomerate while the polymer solution is still fluid. As a consequence the membrane will have a more homogeneous, symmetrical structure. The appropriate temperature of the non-solvent bath can be determined for each specific case with routine experiments.

Pore forming agents are generally at least partially, if not completely, removed from the membrane in the non-solvent bath in step (iii)

Once removed from the precipitation bath the membrane may undergo additional treatments, for instance rinsing. As a last step the membrane is typically dried.

As stated above, membranes (ME) comprising a polymer (PSI) as defined above are antithrombogenic; in particular, it has been observed that membranes (ME) comprising polymers (PSI) of the present invention have a higher antithrombogenic effect than membranes comprising a corresponding unmodified aromatic sulfone polymer. Furthermore, even after washing steps that remove all or most of the pore-forming agent, permeability and wettability remain high. Therefore, in a preferred embodiment, method (MPUR) comprises the use of a membrane (ME) comprising at least one polymer (PSI) as defined above, said membrane being free from pore-forming agents, in particular from PVP. A membrane (ME) free from pore-forming agents can be obtained:
from a polymer solution (SP) as defined above, said solution (SP) being free from pore-forming agents, in particular, free from PVP; or
by subjecting to a washing step a membrane (ME) obtained from a polymer solution (SP) comprising at least one polymer (PSI) as defined above, a polar solvent (S) and a pore-forming agent. The washing step is typically carried out with hot water, usually at a temperature ranging from 40° C. to 90° C., preferably from 70° C. to 90° C., more preferably at 80° C., or with steam at a temperature ranging from 110° C. to 135° C., or with a hypochlorite solution at room temperature.

A membrane (ME) comprising at least one polymer (PSI) as defined above, said membrane being free from pore-forming agents, is a further aspect of the present invention.

A polymer solution (SP) comprising at least one polymer (PSI) as defined above and a polar solvent (S), said solution (SP) being free from pore-forming agents is a further aspect of the present invention.

For the avoidance of doubt, the expression “free from pore-forming agent” means that the weight amount of the pore-forming agent with respect to the overall weight of membrane (ME) or of solution (SP) is less than 0.1% wt or ranges from 0 to 0.1% wt; preferably, the amount is less than 0.09% wt., less than 0.05% wt. or the amount is 0%.

For the above reason, membranes (ME) are advantageously used in a method (MPUR) wherein the biological fluid is a blood product, said method (MPUR) being carried out in an extracorporeal circuit.

In a further aspect, membranes (ME) can be advantageously used for treating a subject suffering from impaired kidney function, the method comprising subjecting a patient to a procedure selected from haemodialysis, hemofiltration, hemoconcentration or hemodiafiltration, said procedure being carried out with a filtering device comprising a bundle of hollow fibers of membranes (ME), preferably membranes (ME) having an average pore diameter of from 0.001 to 5 μm.

Should the disclosure of any patents, patent applications, and publications which are incorporated herein by reference conflict with the description of the present application to the extent that it may render a term unclear, the present description shall take precedence.

The invention will be now described in more details with reference to the following examples, whose purpose is merely illustrative and not intended to limit the scope of the invention.

### Raw Materials

PSI is a polysulfone isosorbide polymer of molecular formula:

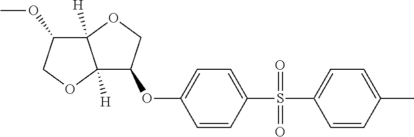

possessing a M_{w} of between 94 000 and 99 000, and a polydispersity index of 1.7 to 1.8, available under the form of prills or “soft pellets”; before being used for the preparation of the dope solutions, PSI was dried in oven for 2 hours at 50° C., so as to remove moisture.

VERADEL® 3000 MP polyethersulfone (PESU) produced by Solvay Specialty Polymers.

N-methyl pirrolidone (NMP), dimethyl acetamide (DMAc) and isopropyl alcohol (IPA) were obtained from Sigma Aldrich®

### General Procedure for the Manufacture of Solution (SP) of Sulfone Polymer for Membrane Manufacture.

Solutions (SP) comprising the ingredients listed in Table 1 were prepared by mixing the selected polymer, the solvent and, optionally, the pore-forming agent for a time ranging from 30 minutes to 6 hours in a temperature range from 25° C. to 50° C.

Ingredients are listed in the following Table 1:

TABLE 1

| Solution | | | |
|---|---|---|---|
| (SP) | Polymer | Additives | Solvent |
| SP-1 | PSI (15% w/w) | PVP K90 (5% w/w) | NMP (80% w/w) |
| SP-1C* | VERADEL ® | PVP K90 (5% w/w) | NMP (80% w/w) |
| | 3000 P (PESU) | | |
| | (15% w/w) | | |
| SP-2 | PSI (20% w/w) | — | DMAc (80%) |
| SP2-C | VERADEL ® | — | DMAc (80%) |
| | 3000 MP 20% | | |
| | w/w (PESU) | | |

*In this and in the following tables, C stands for “comparative”

### Preparation of Porous Membranes

Flat sheet porous membranes were prepared by filming solutions SP1 and SP1C over a suitable smooth glass support by means of an automatized casting knife. Membrane casting was performed by keeping the dope solutions, the casting knife and the support temperatures at 25° C., so as to prevent premature precipitation of the polymer. The knife gap was set to 250 μm. After casting, films of porous membranes (ME) were obtained and were immediately immersed in a coagulation bath in order to induce phase inversion. The coagulation bath consisted of pure de-ionized water. After coagulation, the membranes were washed several times in pure water during the following days to remove residual solvent traces. The membranes were stored (wet) in water.

### Water Flux Permeability Measurements:

Water flux (J) through each membrane at given pressure, is defined as the volume which permeates per unit area and per unit time. The flux is calculated by the following equation:

$$J = \frac{V}{A Δ t}$$

V (I) is the volume of permeate, A (m²) is the membrane area, and Δt (h) is the operation time. J is hence measured in I/(h×m²).

Water flux measurements were conducted at room temperature (23° C.) using a cross-flow configuration under a constant pressure of 1 bar. Results are summarized in Table 2a here below.

TABLE 2a

| | Permeability |
|---|---|
| Membrane** | l/(h × m²) |
| ME-1 | 700 ± 30 |
| ME-1 C | 480 ± 5 |

**ME-1 was obtained from dope solution SP-1, while membrane ME-1C was obtained from dope solution SP-1C.

The data reported in Table 2a demonstrate that membrane ME-1, obtained from dope solution SP-1, which comprises a PSI polymer, is more permeable to water than membrane ME-1C, comprising a PES polymer.

Membranes ME-1 and ME-1C were subjected to washing treatments with water at 80° C. for 6 hours and with a 4000 ppm NaOCI water solution for 6 hours in order to remove the PVP, then permeability was measured. The results are reported in Table 2b here below.

TABLE 2b

| | | Permeability |
|---|---|---|
| | Permeability | l/(h × m²) after washing |
| | l/(h × m²) after washing | with a 4000 ppm NaOCl |
| Membrane | with water (80° C./6 hrs) | water solution for 6 hours |
| ME-1 | 550 ± 20 | 1600 ± 20 |
| ME-1C | 410 ± 10 | 1240 ± 60 |

The results reported in Table 2b above demonstrate that even after washings treatments and removal of PVP, the water permeability of membrane ME-1 is higher than that of membrane ME-1C.

### Gravimetric Porosity Measurements

Membrane porosity (εₘ) was determined according to the gravimetric method, as detailed below. Perfectly dry membrane pieces were weighed and impregnated in isopropylic alcohol (IPA) for 24 h; after this time, the excess of the liquid was removed with tissue paper, and membranes weight was measured again. Finally, from the dry and the wet weight of the sample, it is possible to evaluate the porosity of the membrane using the following formula

$${ε}_{m} ( \% ) = \frac{Ww - Wd / ρ w}{Ww - Wd / ρ w + ( \frac{Wd}{ρ P} )} \times 100$$

where W_{w} is the weight of the wet membrane, W_{d} is the weight of the dry membrane, ρ_{w} is the IPA density (0.785 g/cm³) and ρₚ is the polymer density (equal to 1.37 g/cm³ for the polymer (PSI) used). For all membranes types, at least three measurements were performed; then, average values and corresponding standard deviations were calculated. Table 3 below reports the results of gravimetric porosity measurements carried out on membranes ME-1 and ME-1C as such and after washings treatments with water at 80° C. for 6 hours and with a 4000 ppm NaOCl water solution for 6 hours.

TABLE 3

| | Porosity (%) | Porosity | Porosity (%) |
|---|---|---|---|
| | of the | (%) after | after washing |
| | membrane | washing with | with a 4000 ppm NaOCl |
| Membrane | as such | water (80° C./6 hrs) | water solution for 6 hours |
| ME-1 | 90 ± 1 | 89 ± 1 | 89 ± 1 |
| ME-1C | 89 ± 1 | 89 ± 1 | 89 ± 1 |

The results reported in Table 3 demonstrate that, after washings and removal of PVP, the porosity of the membranes remains substantially unchanged.

### Contact Angle Measurements

Static contact angles (SCA) versus water (5 μL droplets) of porous membranes ME-1 and ME-1C were measured on the membranes as such and after washing with water (80° C./6 hrs). Measurement were carried out with a DSA10 apparatus manufactured by Krüss GmbH, Germany. The results are reported in Table 4.

TABLE 4

| | SCA (water) | SCA (water) after washing |
|---|---|---|
| Membrane | without washing | with water (80° C./6 hrs) |
| ME-1 | 42.2 ± 2.7 | 48.6 ± 4.0 |
| ME-1C | 51.3 ± 1.7 | 56.5 ± 3.8 |

The results show that contact angles of membrane ME-1 vs water are always than contact angles of membrane ME-1C, before and after washing with water. Therefore, membranes ME-1 are more wettable than membranes ME-1C.

### Preparation of Non-Porous Dense Films (F)

Non-porous, flat dense polymeric films for the performance of blood coagulation tests were prepared from dope solutions SP-2 and SP-2C and by filming each dope solution over a suitable smooth glass support by means of an automatized casting knife at 40° C. The knife gap was set at 500 μm. After casting the films, the solvent was allowed to evaporate in a vacuum oven at 130° C. for 4 hours.

### Blood Coagulation Tests (Determination of the Partial Thromboplastin Time)

Partial thromboplastin time of blood contacted with non-porous dense films was evaluated (in duplicate) according to F2382-04 (Reapproved 2010) [Standard Test Method for Assessment of Intravascular Medical Device Materials on Partial Thromboplastin Time (PTT)].

4 cm² (2×2 cm) specimens of non-porous dense membrane films obtained from dope solutions SP-2 and SP-2C [herein after respectively referred to as (ME-2) and (ME-2C)] were sterilized with 30-35 kGy and covered with 1 ml of citrated plasma, then incubated at 37° C. for 15 minutes. After incubation, the test specimens were contacted with a solution of rabbit brain cefalin (RCB) and with a solution of CaCl.

Average PPT was evaluated on the test and also on polypropylene tubes contacted with 1 ml plasma (negative controls), 4 mm glass beads (positive controls) and natural rubber (biomaterial reference; moderate coagulation activator). The clotting time values for the positive control, for the biomaterial reference control and of the specimens obtained from the aforementioned dope solutions was calculated as percent of the negative control using the following equation:

$$\% Negative Control = ( \frac{Average clotting time ( s ) ofsample )}{( Average clotting time ( s ) of negative controls} ) \times 100$$

The results are reported in Table 5 below.

TABLE 5

| Membrane | PTT |
|---|---|
| ME-2 | 96 |
| ME-2C | 88.4 |

The % negative control value of specimens obtained from a ME-2C, was 88.4, while the value obtained for the specimens obtained from a ME-2 was 96%. By comparing these percentages with the test acceptance criteria reported in F2382-04, it can be appreciated that both sulfone polymers are minimal coagulation activators, but the (PSI) induces less coagulation then the PES.

1-11. (canceled)

12. A purification method (MPUR) for a biological fluid comprising at least a filtration step through a membrane (ME) obtained from a sulfone polymer (PSI) having recurring units, wherein more than 50% moles, with respect to all the recurring units of the sulfone polymer (PSI), are recurring units (R_{PSI}) selected from the group consisting of those of formulae (R_{PSI}-1) and (R_{PSI}-2) herein below: 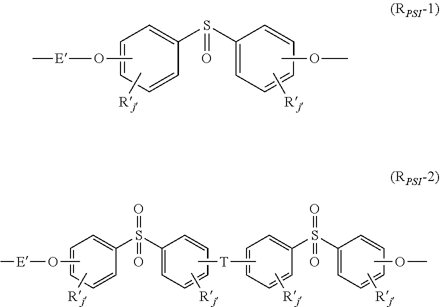
wherein:
each of E′, equal to or different from each other and at each occurrence, is selected from the group consisting of those of formulae (E′-I) to (E′-III): 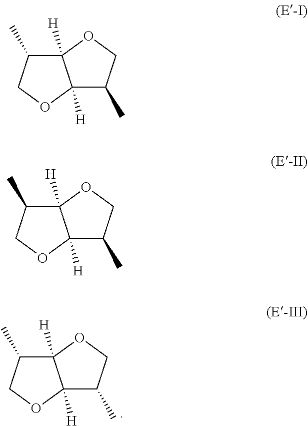
each R′ is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium;
j′ is zero or an integer of 1 to 4; and
T is selected from the group consisting of a bond, —CH₂—, —C(O)—, —C(CH₃)₂—, —C(CF₃)₂—, —C(═CCl₂)—, —C(CH₃)(CH₂CH₂COOH)—, and a group of formula:

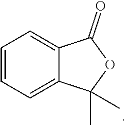

13. The method of claim 12, wherein the membrane (ME) comprising an amount of pore-forming agent of less than 0.1% wt., with respect to the overall weight of the membrane (ME).

14. The method of claim 12, wherein the recurring units (R_{PSI}) of the sulfone polymer (PSI) are recurring units of any formula selected from the group consisting of formulae (R_{PSI}-1a), (R_{PSI}-1b), (R_{PSI}-1c), (R_{PSI}-2a), (R_{PSI}-2b), and (R_{PSI}-2c): 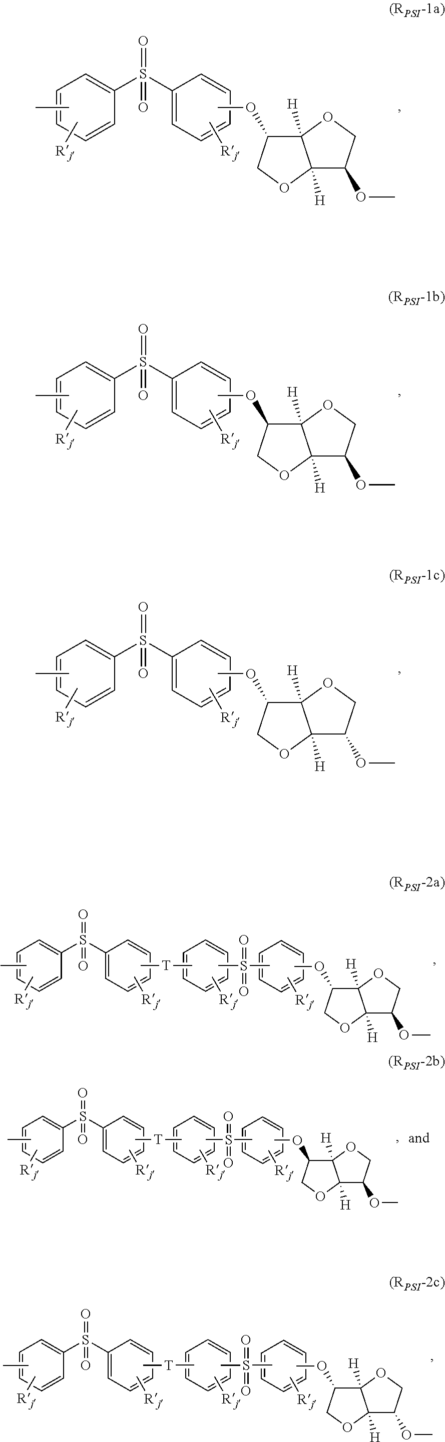
wherein R′, J′ and T have the meaning as defined in claim 12.

15. The method of claim 14, wherein the recurring units (R_{PSI}) of the sulfone polymer (PSI) are recurring units of formula (R_{PSI}-1a) and (R_{PSI}-2a), optionally in combination with recurring units of formula (R_{PSI}-1b), (R_{PSI}-2b), (R_{PSI}-1c) and (R_{PSI}-2c).

16. The method of claim 15, wherein the recurring units (R_{PSI}) of the sulfone polymer (PSI) are the recurring units of formula (R_{PSI}-1a), optionally in combination with the recurring units of formula (R_{PSI}-1b) and (R_{PSI}-1c).

17. The method of claim 12, wherein the membrane (ME) is in the form of:
a flat sheet;
a tubular membrane having a diameter greater than 3 mm;
a capillary membrane having a diameter comprised between 0.5 mm and 3 mm; or
a hollow fiber having a diameter of less than 0.5 mm.

18. The method of claim 12, wherein the biological fluid is selected from the group consisting of a blood product, urine, saliva and interstitial fluids.

19. The method of claim 12, which is carried out by means of an extracorporeal circuit when the biological fluid is blood.

20. The method of claim 19, wherein the extracorporeal circuit comprises a hemodialyzer, which comprises the membrane (ME) in the form of a cylindrical bundle of hollow fibers having a diameter of less than 0.5 mm.

21. A membrane [membrane (ME)] obtained from a sulfone polymer (PSI) respect to all the recurring units of the sulfone polymer (PSI), are recurring units (R_{PSI}) selected from the group consisting of those of formulae (R_{PSI}-1) and (R_{PSI}-2) herein below: 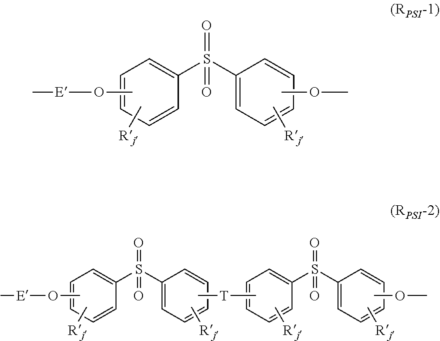
wherein:
each of E′, equal to or different from each other and at each occurrence, is selected from the group consisting of formulae (E′-I) to (E′-III): 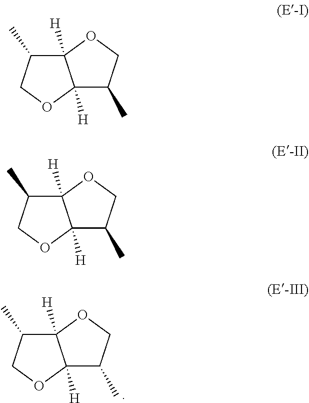
each R′ is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium;
j′ is zero or an integer of 1 to 4; and
T is selected from the group consisting of a bond, —CH₂—, —C(O)—, —C(CH₃)₂—, —C(CF₃)₂—, —C(═CCl₂)—, —C(CH₃)(CH₂CH₂COOH)—, and a group of formula: 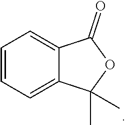
said membrane (ME) comprising an amount of pore-forming agent of less than 0.1% wt with respect to the overall weight of the membrane (ME).

22. A method for preparing a membrane (ME) free from pore forming agents, comprising the following steps:
preparing a polymer solution (SP) comprising at least one sulfone polymer (PSI) and a polar solvent (S);
processing said polymer solution (SP) into a film; and
contacting said film with a non-solvent bath to induce the precipitation of the sulfone polymer (PSI) from the polymer solution (SP) and to form the membrane (ME);
wherein either
1) the polymer solution (SP) comprises less than 0.1 wt % pore-forming agents, with respect to the overall weight of solution (SP), or
2) the polymer solution (SP) comprises from 0.1 to 40% by weight, with respect to the overall weight of the solution (SP), of a pore forming agent which is selected from polyvinylpyrrolidone (PVP) and polyethyleneglycol (PEG) having a molecular weight of at least 200;
wherein
when the polymer solution (SP) comprises from 0.1% to 40% by weight of the pore forming agent under 2), the method further comprises a step of washing the membrane (ME),
wherein the sulfone polymer (PSI) has recurring units, of which more than 50% moles, with respect to all the recurring units of the sulfone polymer (PSI), are recurring units (R_{PSI}) selected from the group consisting of those of formulae (R_{PSI}-1) and (R_{PSI}-2) herein below: 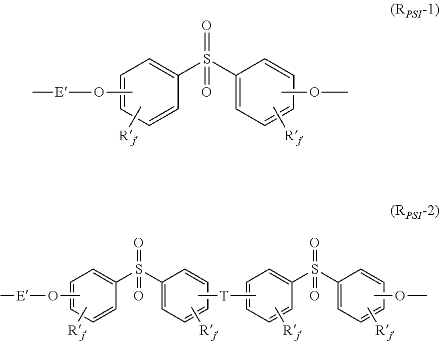
wherein:
each of E′, equal to or different from each other and at each occurrence, is selected from the group consisting of those of formulae (E′-I) to (E′-III): 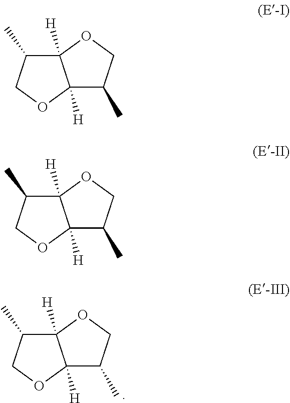
each R′ is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium; and
j′ is zero or an integer of 1 to 4;
T is selected from the group consisting of a bond, —CH2-, —C(O)—, —C(CH3)2-, —C(CF3)2-, —C(═CCl2)-, —C(CH3)(CH2CH2COOH)—, and a group of formula:

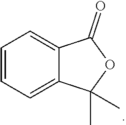

23. The method of claim 22, wherein the step of processing of the polymer solution (SP) into a film is performed by casting or wet-spinning.

24. The method of claim 22, wherein the non-solvent bath contains a non-solvent selected from the group consisting of: water, aliphatic alcohols having from 1 to 6 carbon atoms, and mixture thereof.

25. The method of claim 24, wherein the non-solvent bath comprises, in addition to the non-solvent, up to 40% by weight, with respect to the total weight of the non-solvent bath, of a solvent for the sulfone polymer (PSI).

26. The method of claim 22, wherein, when 2) the polymer solution (SP) comprises from 0.1% to 40% by weight of the pore forming agent, the washing step is carried out with hot water at a temperature ranging from 40° C. to 90° C., or with steam at a temperature ranging from 110° C. to 135° C., or with a hypochlorite solution at room temperature.

27. The method of claim 22, wherein the membrane (ME) is in the form of:
a flat sheet;
a tubular membrane, said tubular membranes being possibly a tubular membrane having a diameter greater than 3 mm;
a capillary membrane having a diameter comprised between 0.5 mm and 3 mm; or
a hollow fiber having a diameter of less than 0.5 mm.

28. The method of claim 22, wherein either
the film is flat in shape when the membrane (ME) is a flat membrane, or
the film is tubular in shape when the membrane (ME) is a tubular or hollow fiber membrane.

29. The method of claim 22, wherein the overall concentration of the sulfone polymer (PSI) in the polymer solution (SP) is at least 12% by weight and at most 40% by weight, based on the total weight of the polymer solution (SP).

30. The method of claim 22, wherein the recurring units (R_{PSI}) of the sulfone polymer (PSI) are recurring units of any formula selected from the group consisting of formulae (R_{PSI}-1a), (R_{PSI}-1b), (R_{PSI}-1c), (R_{PSI}-2a), (R_{PSI}-2b), and (R_{PSI}-2c): 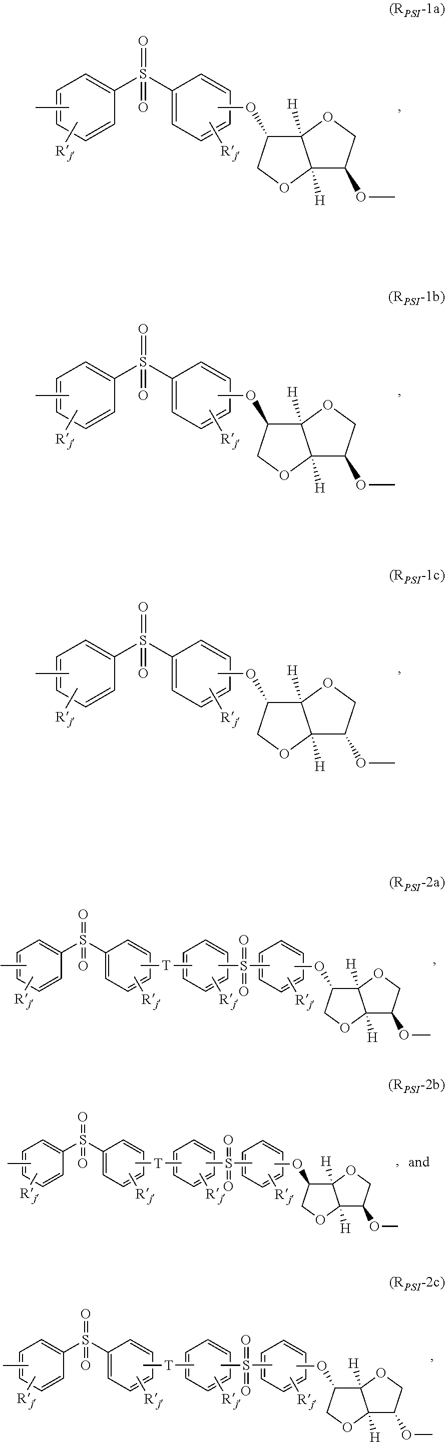
wherein R′, J′ and T have the meaning as defined in claim 22.

31. The method of claim 29, wherein the recurring units (R_{PSI}) of the sulfone polymer (PSI) are the recurring units of formula (R_{PSI}-1a), optionally in combination with the recurring units of formula (R_{PSI}-1 b) and (R_{PSI}-1c).
